(19) United States
(12) Patent Application Publication
(10) Pub. No.: US 2024/0407853 A1
(43) Pub. Date: Dec. 12, 2024

(54) ENDOSCOPE CLEANING SHEATH WITH INTEGRAL POSITION SENSOR

(71) Applicants: Acclarent, Inc., Irvine, CA (US); Biosense Webster (Israel) LTD., Yokneam (IL)

(72) Inventors: Cesar Fuentes-Ortega, Pasadena, CA (US); Henry F. Salazar, Pico Rivera, CA (US); Shubhayu Basu, Anaheim, CA (US); Uriel Hod, Haifa (IL); Raymond Yue-sing Tang, Rosemead, CA (US); Gigi Koe, Anaheim, CA (US); Hao H. Tran, Santa Ana, CA (US); Priyadarshini Ghosh, Irvine, CA (US); Zachary Radner, Anaheim, CA (US)

(21) Appl. No.: 18/735,472

(22) Filed: Jun. 6, 2024

Related U.S. Application Data
(60) Provisional application No. 63/471,798, filed on Jun. 8, 2023.

(51) Int. Cl. A61B 34/20; A61B 1/00; A61B 1/12

(52) U.S. Cl. CPC A61B 34/20; A61B 1/00009; A61B 1/00097; A61B 1/00105; A61B 1/00128; A61B 1/00135; A61B 1/126; A61B 2034/2051; A61B 2034/2074

(57) ABSTRACT

An apparatus for use with an endoscope or other ENT instrument includes a hub, a shaft assembly, and one or more navigation sensors. The hub being configured to receive a portion of the endoscope and including a fluid port. The shaft assembly extends from the hub and defines a hollow interior. The hollow interior is configured to receive a tubular portion of the endoscope and communicate fluid within the shaft assembly to an open distal end of the shaft assembly. At least one of the one or more navigation sensors is disposed proximate the distal end of the shaft assembly. The one or more navigation sensors are configured to communicate with an IGS navigation system to locate at least a portion of the shaft assembly in three dimensional space.

The present application claims priority under 35 U.S.C. § 119 to U.S. Patent Application Ser. No. 63/471,798, filed Jun. 8, 2023 and having the title “ENDOSCOPE CLEANING SHEATH WITH INTEGRAL POSITION SENSOR,” which is herein incorporated by reference in its entirety.

### BACKGROUND

Image-guided surgery (IGS) is a technique where a computer is used to obtain a real-time correlation of the location of an instrument that has been inserted into a patient's body to a set of preoperatively obtained images (e.g., a CT or MRI scan, 3-D map, etc.), such that the computer system may superimpose the current location of the instrument on the preoperatively obtained images. An example of an electromagnetic IGS navigation system that may be used in IGS procedures is the CARTO® 3 System by Biosense-Webster, Inc., of Irvine, California. In some IGS procedures, a digital tomographic scan (e.g., CT or MRI, 3-D map, etc.) of the operative field is obtained prior to surgery. A specially programmed computer is then used to convert the digital tomographic scan data into a digital map. During surgery, some instruments can include sensors (e.g., electromagnetic coils that emit electromagnetic fields and/or are responsive to externally generated electromagnetic fields), which can be used to perform the procedure while the sensors send data to the computer indicating the current position of each sensor-equipped instrument. The computer correlates the data it receives from the sensors with the digital map that was created from the preoperative tomographic scan. The tomographic scan images are displayed on a video monitor along with an indicator (e.g., crosshairs or an illuminated dot, etc.) showing the real-time position of each surgical instrument relative to the anatomical structures shown in the scan images. The surgeon is thus able to know the precise position of each sensor-equipped instrument by viewing the video monitor even if the surgeon is unable to directly visualize the instrument itself at its current location within the body.

In some instances, it may be desirable to guide an instrument using IGS techniques described above. However, certain IGS navigation systems may be equipped for use with specific instruments and certain instruments may be equipped for use with specific IGS navigation systems or no IGS navigation system at all. Thus, it may be desirable to provide an easily implemented and cost-effective means to incorporate compatibility with a given IGS navigation system into a variety of instruments. While several systems and methods have been made and used in connection with IGS navigation stems, it is believed that no one prior to the inventors has made or used the invention described in the appended claims.

### BRIEF DESCRIPTION OF THE DRAWINGS

The drawings and detailed description that follow are intended to be merely illustrative and are not intended to limit the scope of the invention as contemplated by the inventors.

FIG. 1 is a schematic view of an example of a surgery navigation system being used on a patient seated in an example of a medical procedure chair;

FIG. 2 is a perspective view of an example of an instrument with a cleaning sleeve removably secured to the instrument;

FIG. 3 is a perspective view of the instrument and the cleaning sleeve of FIG. 2, with the cleaning sleeve removed from the instrument;

FIG. 4 is a side cross-sectional view of the instrument and the cleaning sleeve of FIG. 2, the cross-section being taken along line 4-4 of FIG. 2;

FIG. 5 is a perspective view of another example of a cleaning sleeve for use with the instrument of FIG. 2;

FIG. 6 is an exploded perspective view of a hub of the cleaning sleeve of FIG. 5;

FIG. 7 is a side cross-sectional view of the cleaning sleeve of FIG. 5, the cross-section taken along line 7-7 of FIG. 5;

FIG. 8 is a perspective view of a cap of the hub of FIG. 6;

FIG. 9 is a front elevational view of the cap of FIG. 8;

FIG. 10 is a partial perspective view of yet another example of a cleaning sleeve for use with the instrument of FIG. 2;

FIG. 11 is an exploded perspective view of a hub of the cleaning sleeve of FIG. 10;

FIG. 12 is a side cross-sectional view of the cleaning sleeve of FIG. 10, the cross-section taken along line 12-12 of FIG. 10;

FIG. 13 is a partial perspective view of still another example of a cleaning sleeve for use with the instrument of FIG. 2;

FIG. 14A is a side cut-away view of the cleaning sleeve of FIG. 13 being inserted onto the instrument of FIG. 2, a hub of the cleaning sleeve being positioned proximate a portion of the instrument;

FIG. 14B is another side cut-away view of the cleaning sleeve of FIG. 14 being inserted onto the instrument of FIG. 2, the hub of the cleaning sleeve being actuated to engage the instrument;

FIG. 14C is yet another side cut-away view of the cleaning sleeve of FIG. 14 being inserted onto the instrument of FIG. 2, the hub of the cleaning sleeve engaging the instrument;

FIG. 15 is a partial perspective view of still another example of a cleaning sleeve for use with the instrument of FIG. 2;

FIG. 16 is a side exploded cross-sectional view of the cleaning sleeve of FIG. 15, the cross-section taken along line 16-16 of FIG. 15.

FIG. 17 is another side cross-sectional view of the cleaning sleeve of FIG. 15 with the instrument of FIG. 2 inserted therein; and

FIG. 18 is a front cross-sectional view of the cleaning sleeve of FIG. 15 with the instrument of FIG. 2 inserted therein, the cross-section taken along line 18-18 of FIG. 15.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

For clarity of disclosure, the terms “proximal” and “distal” are defined herein relative to a surgeon, or other operator, grasping a surgical instrument having a distal surgical end effector. The term “proximal” refers to the position of an element arranged closer to the surgeon, and the term “distal” refers to the position of an element arranged closer to the surgical end effector of the surgical instrument and further away from the surgeon. Moreover, to the extent that spatial terms such as “upper,” “lower,” “vertical,” “horizontal,” or the like are used herein with reference to the drawings, it will be appreciated that such terms are used for exemplary description purposes only and are not intended to be limiting or absolute. In that regard, it will be understood that surgical instruments such as those disclosed herein may be used in a variety of orientations and positions not limited to those shown and described herein.

As used herein, the terms “about” and “approximately” for any numerical values or ranges indicate a suitable dimensional tolerance that allows the part or collection of components to function for its intended purpose as described herein.

### I. Example of an Image Guided Surgery Navigation System

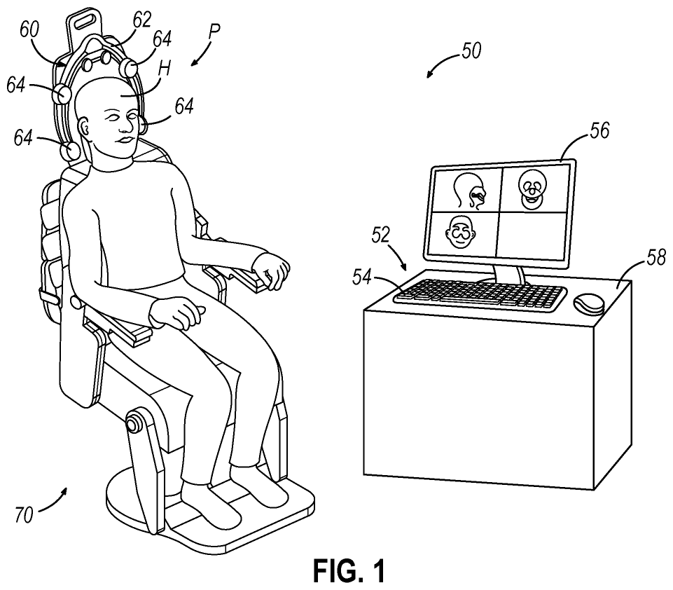

When performing a medical procedure within a head (H) of a patient (P), it may be desirable to have information regarding the position of an instrument within the head (H) of the patient (P), particularly when the instrument is in a location where it is difficult or impossible to obtain an endoscopic view of a working element of the instrument within the head (H) of the patient (P). FIG. 1 shows an example of an IGS navigation system 50 enabling an ENT procedure to be performed using image guidance. In addition to or in lieu of having the components and operability described herein, the IGS navigation system 50 may be constructed and operable in accordance with at least some of the teachings of U.S. Pat. No. 7,720,521, entitled “Methods and Devices for Performing Procedures within the Ear, Nose, Throat and Paranasal Sinuses,” issued May 18, 2010, the disclosure of which is incorporated by reference herein, in its entirety; and U.S. Pat. Pub. No. 2014/0364725, entitled “Systems and Methods for Performing Image Guided Procedures within the Ear, Nose, Throat and Paranasal Sinuses,” published Dec. 11, 2014, now abandoned, the disclosure of which is incorporated by reference herein, in its entirety.

The IGS navigation system 50 of the present example comprises a field generator assembly 60, which comprises set of magnetic field generators 64 that are integrated into a horseshoe-shaped frame 62. The field generators 64 are operable to generate alternating magnetic fields of different frequencies around the head (H) of the patient (P). An instrument, such as any of the instruments described below, may be inserted into the head (H) of the patient (P). Such an instrument may be a standalone device or may be positioned on an end effector. In the present example, the frame 62 is mounted to a chair 70, with the patient (P) being seated in the chair 70 such that the frame 62 is located adjacent to the head (H) of the patient (P). By way of example only, the chair 70 and/or the field generator assembly 60 may be configured and operable in accordance with at least some of the teachings of U.S. Pat. No. 10,561,370, entitled “Apparatus to Secure Field Generating Device to Chair,” Issued Feb. 18, 2020, the disclosure of which is incorporated by reference herein, in its entirety.

The IGS navigation system 50 of the present example further comprises a processor 52, which controls the field generators 64 and other elements of the IGS navigation system 50. For instance, the processor 52 is operable to drive the field generators 64 to generate alternating electromagnetic fields; and process signals from the instrument to determine the location of a navigation sensor or position sensor in the instrument within the head (H) of the patient (P). The processor 52 comprises a processing unit (e.g., a set of electronic circuits arranged to evaluate and execute software instructions using combinational logic circuitry or other similar circuitry) communicating with one or more memories. The processor 52 of the present example is mounted in a console 58, which comprises a number of operating controls 54 that include a keypad and/or a pointing device such as a mouse or trackball. A physician uses operating the controls 54 to interact with the processor 52 while performing the surgical procedure.

While not shown, the instrument may include a navigation sensor or position sensor that is responsive to positioning within the alternating magnetic fields generated by the field generators 64. A coupling unit (not shown) may be secured to the proximal end of the instrument and may be configured to provide communication of data and other signals between the console 58 and the instrument. The coupling unit may provide wired or wireless communication of data and other signals.

In some versions, the navigation sensor or position sensor of the instrument may comprise at least one coil at or near the distal end of the instrument. When such a coil is positioned within an alternating electromagnetic field generated by the field generators 64, the alternating magnetic field may generate electrical current in the coil, and this electrical current may be communicated along the electrical conduit(s) in the instrument and further to the processor 52 via the coupling unit. This phenomenon may enable the IGS navigation system 50 to determine the location of the distal end of the instrument within a three-dimensional space (i.e., within the head (H) of the patient (P), etc.). To accomplish this, the processor 52 executes an algorithm to calculate location coordinates of the distal end of the instrument from the position related signals of the coil(s) in the instrument. Thus, a navigation sensor may serve as a position sensor by generating signals indicating the real-time position of the sensor within three-dimensional space.

The processor 52 uses software stored in a memory of the processor 52 to calibrate and operate the IGS navigation system 50. Such operation includes driving the field generators 64, processing data from the instrument, processing data from the operating controls 54, and driving the display screen 56. In some implementations, operation may also include monitoring and enforcement of one or more safety features or functions of the IGS navigation system 50. The processor 52 is further operable to provide video in real time via a display screen 56, showing the position of the distal end of the instrument in relation to a video camera image of the patient's head (H), a CT scan image of the patient's head (H), and/or a computer-generated three-dimensional model of the anatomy within and adjacent to the patient's nasal cavity. The display screen 56 may display such images simultaneously and/or superimposed on each other during the surgical procedure. Such displayed images may also include graphical representations of instruments that are inserted in the patient's head (H), such that the operator may view the virtual rendering of the instrument at its actual location in real time. By way of example only, the display screen 56 may provide images in accordance with at least some of the teachings of U.S. Pat. No. 10,463,242, entitled “Guidewire Navigation for Sinuplasty,” issued Nov. 5, 2019, the disclosure of which is incorporated by reference herein, in its entirety. In the event that the operator is also using an endoscope, the endoscopic image may also be provided on the display screen 56.

The images provided through the display screen 56 may help guide the operator in maneuvering and otherwise manipulating instruments within the patient's head (H). It should also be understood that other components of a surgical instrument and other kinds of surgical instruments, as described below, may incorporate a navigation sensor like the navigation sensor described above.

II. Example of a Cleaning Sleeve for Use with One or More Endoscopic Instruments

In some instances, it may be desirable to provide an adapter or other component having one or more integral navigation sensors, where such an adapter may be readily coupled with an instrument that otherwise lacks any navigation sensors. In such a scenario, the adapter may impart navigation capabilities to the instrument. Similarly, it may be desirable to use an adapter having one or more integral navigation sensors in combination with an instrument that already has one or more navigation sensors, where the position data from the one or more navigation sensors of the adapter may supplement the position data from the one or more navigation sensors of the instrument. In such a scenario, the adapter may enhance navigation capabilities to the instrument. In either of the above scenarios, the adapter may be configured to avoid adding bulk to the instrument; and to be easily assembled with the instrument in the surgical field or operating room. Moreover, the adapter may be configured to accommodate different kinds of instruments and/or instruments otherwise having different outer diameters, such that the adapter need not necessarily be limited in its compatibility to just one single instrument size or type.

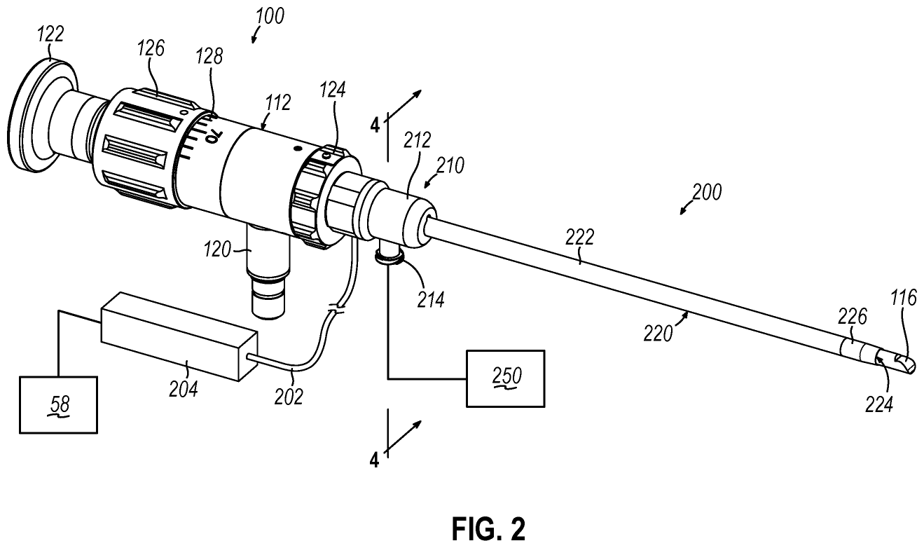

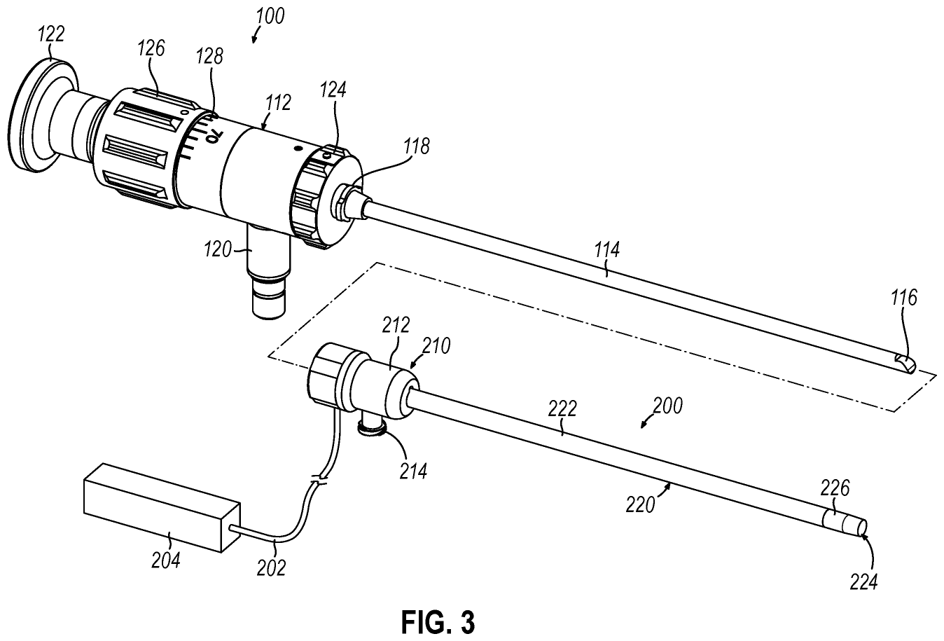

FIGS. 2-3 show an example of an adapter, that may provide the above-described benefits and functionality, in the form of a cleaning sleeve 200. In the present example, the cleaning sleeve 200 is coupled with an endoscope 100. As will be described in greater detail below, cleaning sleeves similar to the cleaning sleeve 200 of the present example may be used with endoscopes similar to the endoscope 100 to clean one or more components of the endoscope during use thereof. However, existing endoscopes lack features such as one or more integral navigation sensors to facilitate navigation of such endoscopes via systems such as the IGS navigation system 50. Therefore, it may be desirable to add navigation capabilities to such endoscopes via components such as cleaning sleeves. Such a combination may be desirable to facilitate the use of certain virtual reality features and/or advanced mapping features. Additionally, such a combination may be desirable to facilitate real-time overlay of positional data on images, providing true anatomical measurements that may improve workflows and/or procedure outcomes. Although certain combinations of adaptors and instruments are described herein, it should be understood that such combinations may be combined with other adaptors and/or instruments to promote use of the IGS navigation system 50 with multiple instruments used in a given procedure.

The endoscope 100 of the present example is generally configured to provide visualization within an anatomical passageway (e.g., within the nasal cavity, etc.). The endoscope 100 includes a body 112 and a shaft 114 extending distally from the body 112. The distal end of the shaft 114 includes a curved transparent window 116. Although not shown, it should be understood that a plurality of rod lenses and light transmitting fibers may extend along the length of the shaft 114. A lens is positioned at the distal end of the rod lenses and a swing prism is positioned between the lens and the window 116. The swing prism is pivotable about an axis that is transverse to the longitudinal axis of the shaft 114. The swing prism defines a line of sight that pivots with the swing prism. The line of sight defines a viewing angle relative to the longitudinal axis of the shaft 114. This line of sight may pivot from approximately 0 degrees to approximately 120 degrees, from approximately 10 degrees to approximately 90 degrees, or within any other suitable range. The swing prism and the window 116 also provide a field of view spanning approximately 60 degrees (with the line of sight centered in the field of view). Thus, the field of view provides a viewing range spanning approximately 180 degrees, approximately 140 degrees, or any other range, based on the pivot range of the swing prism. Of course, all of these values are merely examples.

The body 112 of the present example includes a light post 120, an eyepiece 122, a rotation dial 124, a pivot dial 126, and an accessory attachment portion 118. The light post 120 is in communication with the light transmitting fibers in the shaft 114 and is configured to couple with a source of light to thereby illuminate the site in the patient distal to the window 116. The eyepiece 122 is configured to provide visualization of the view captured through the window 116 via the optics of the endoscope 100. It should be understood that a visualization system (e.g., camera and display screen, etc.) may be coupled with the eyepiece 122 to provide visualization of the view captured through the window 116 via the optics of the endoscope 100.

The rotation dial 124 is configured to rotate the shaft 114 relative to the body 112 about the longitudinal axis of the shaft 114. It should be understood that such rotation may be carried out even while the swing prism is pivoted such that the line of sight is non-parallel with the longitudinal axis of the shaft 114.

The pivot dial 126 is coupled with the swing prism and is thereby operable to pivot the swing prism about the transverse pivot axis. Indicia 128 on the body 112 provides visual feedback indicating the viewing angle. Various suitable components and arrangements that may be used to couple rotation the dial 124 with the swing prism will be apparent to those of ordinary skill in the art in view of the teachings herein.

The accessory attachment portion 118 is positioned on a distal end of the body 112 proximate the shaft 114. As will be described in greater detail below, the accessory attachment portion 118 is generally configured to facilitate coupling between the endoscope 100 and certain accessory components that may be used with the endoscope 100. In the present example, the accessory attachment portion 118 defines a flanged configuration to facilitate such coupling. In other examples, the accessory attachment portion 118 may take on a variety of forms, which may include threads, posts, chamfers, bores, and/or etc. In still other examples, the accessory attachment portion 118 is entirely optional and may be omitted.

As best seen in FIGS. 2 and 3, the cleaning sleeve 200 of the present example includes a hub 210, a shaft assembly 220 extending distally from the hub 210, a cable 202, and a connector 204. The cleaning sleeve 200 is generally configured to clean the window 116 of endoscope 100 during a procedure. As will be understood, the cleaning sleeve 200 is configured to direct a flow of fluid through the hub 210 and into the shaft assembly 220 to communicate the fluid to the area proximate the window 116. In other words, the cleaning sleeve 200 is configured to communicate fluid to the area proximate the window 116 to clear obstructions from the window 116 such as tissue, debris, mucous, blood, or other obstructions that may be present during a procedure.

The shaft assembly 220 includes a sleeve 222 extending distally from the hub 210. The sleeve 222 terminates at an open distal end 224. In the present example, the sleeve 222 defines a length less than the length of the shaft 114 of the endoscope 100 such that the window 116 may protrude from the open distal end 224. The sleeve 222 is generally of a rigid configuration but may optionally be configured with at least some flexibility. At least some flexibility of the sleeve 222 may be desirable to promote use of the sleeve 222 with other instruments having differently sized shafts similar to the shaft 114. Although the shaft assembly 220 of the present example is configured with only the sleeve 222, it should be understood that in other examples, the shaft assembly 220 may optionally include multiple sleeves layered together.

The shaft assembly 220 further includes one or more navigation sensors 226. Incorporation of the navigation sensors 226 may be desirable to add navigation functionality to the endoscope 100 via the cleaning sleeve 200 and the IGS navigation system 50. In the present example, at least one navigation sensor 226 is positioned proximate the open distal end 224. Such a positioning may be desirable to facilitate localization of the window 116 due to the proximate relationship between the open distal end 224, the navigation sensor 226, and the window 116. Although not shown, it should be understood that in other examples, additional navigation sensors 226 may be included at various positions along the length of the sleeve 222. Such additional navigation sensors 226 may be desirable to promote enhanced localization of the shaft assembly 220 and/or the other components of cleaning sleeve 200.

The navigation sensors 226 are generally coupled to at least a portion of the sleeve 222. For instance, in some examples, the navigation sensors 226 may be configured as sticker-like sensors that may be adhered to the interior or exterior of the sleeve 222. In multi-sleeve configurations of the shaft assembly 22, the navigation sensors 226 may be layered between two sleeves and secured via adhesive, friction, or both. In yet other examples, the navigation sensors 226 may be integral with a portion of the sleeve 222 with a portion of each navigation sensor 226 being embedded within the structure of the sleeve 222. Still other means of coupling the navigation sensors 226 to the sleeve 222 or other components of the shaft assembly 220 will be apparent to those of ordinary skill in the art in view of the teachings herein.

The navigation sensors 226 are in communication with the connector 204 via the cable 202. As best seen in FIG. 1, such communication is configured to facilitate communication of the navigation sensors 226 with the console of the IGS navigation system 50 via the connector 204. Although not shown, it should be understood that such communication may be facilitated by a plurality of electrical communication paths extending through the sleeve 222 to the open distal end 224. Such electrical communication paths may take on a variety of forms. For instance, in some examples, such electrical communication paths may be configured as a portion of a flexible printed circuit board attached to (e.g., on the inner diameter or outer diameter) or embedded within a portion of the sleeve 222. Such flexible printed circuit boards may additionally include one or more leads to facilitate electrical communication between the wires 202 and/or the connector 204. In other examples, such electrical communication paths may be configured as a plurality of wires extending through the sleeve 222. Such wires may likewise be attached to or embedded within a portion of the sleeve 222. Alternatively, in some examples, the sleeve 222 may be configured to define one or more dedicated lumens configured to receive one or more of such wires. Of course, various other means of facilitating electrical communication paths through the sleeve 222 will be apparent to those of ordinary skill in the art in view of the teachings herein.

The hub 210 is disposed on a proximal end of the shaft assembly 220 and is generally configured to facilitate various components of the instrument 100, and other features associated with the cleaning sleeve 200 to interface with the shaft assembly 220. The hub 210 includes a body 212 and a fluid port 214 extending outwardly from the body 212. As will be described in greater detail below, the fluid port 214 may be in communication with a fluid source 250 to communicate fluid through the shaft assembly 220 and out the open distal end 224 thereof. Although not shown, it should be understood that the hub 210 may also include wire ports and/or conduits to facilitate electrical communication between the cable 202 and the navigation sensors 226.

As will be described in greater detail below, the hub 210 may additionally be configured to removably secure a proximal end of the cleaning sleeve 200 relative to the endoscope 100. Specifically, the hub 210 is configured to removably couple to the accessory attachment portion 118 of the endoscope 100. By way of example only, the hub 210 may provide a snap fit, and interference fit, or any other suitable kind of relationship with the accessory attachment portion 118 endoscope 100. Additionally, in some examples, the hub 210 may be configured to sealingly engage the accessory attachment portion 118. Thus, the hub 210 may additionally include o-rings, seals, gaskets, and/or etc. in some examples. It should be understood that in some versions, such engagement between the hub 210 and the endoscope 100 is entirely optional and may be omitted. For instance, in some examples, the cleaning sleeve 200 may be used with a variety of instruments having different shaft lengths. In such examples, it may still be desirable position features of the cleaning sleeve 200 (e.g., the navigation sensors 226) proximate to specific features of such instruments (e.g., a distal end or end effector). Thus, it may be desirable for the position of the cleaning sleeve 200 relative to the shaft of such instruments to not be tied to particular structural features. In other words, in some examples, a gap may be present between the hub 210 and the body 112 of the endoscope 100. It is therefore not essential that the hub 210 of the cleaning sleeve 200 contact or otherwise engage the body 112 of the endoscope 100. Moreover, some variations of the cleaning sleeve 200 may lack the hub 210.

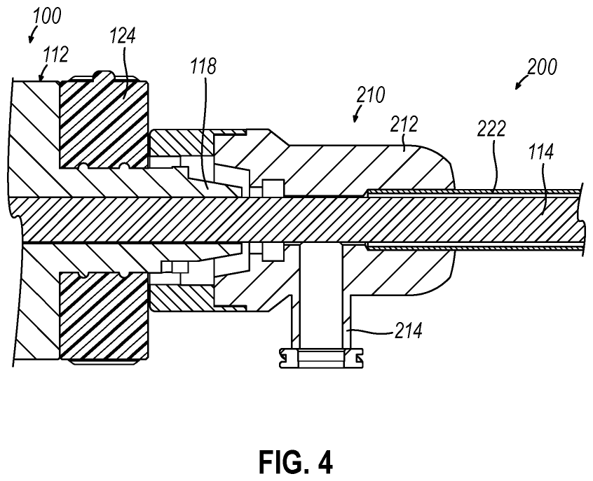

As best seen in FIG. 4, the fluid port 212 is in communication with a hollow interior of the hub 210, which is in communication with a hollow interior of the shaft assembly 220. As can be seen, both the hub 210 and the shaft assembly 220 are configured to provide clearance fit relative to the shaft 114 of the endoscope 100 to define a fluid path between an exterior of the endoscope 100 and an interior of the cleaning sleeve 200 extending from the fluid port 214 through the hub 210 and the shaft assembly 220 to the open distal end 224. Thus, the fluid port 214 may be coupled to the fluid source 250 to communicate fluid through the shaft assembly 220 and out of the open distal end 224. Although the hub 210 of the present example is shown as including a single fluid port 214, it should be understood that in other examples, multiple fluid ports 214 may be used such as in examples of a hub described in greater detail below.

In use, the cleaning sleeve 200 may be used to clean or otherwise remove obstructions from the window 116 of the instrument 100. Specifically, the fluid source 250 may communicate fluid into the fluid port 214 of the hub 210. The fluid source 250 may be configured in a variety of ways to communicate fluid such as a syringe, an automatic or semi-automatic fluid dispenser, and/or etc. Such fluid communication may be continuous or intermittent. For instance, in some examples, fluid communication by the fluid source 250 may be at least partially controlled by an operator. Such operator control may initiate fluid communication by the fluid source 250, after which a fluid communication sequence of continuous flow, intermittent flow, or various combinations thereof may be executed automatically. Alternatively, such operator control may control the entirety of fluid communication by the fluid source 250 (e.g., a fixed flow of fluid communication while an operator input is actuated). Additionally, suitable fluids may include biocompatible fluids such as saline. Suitable fluids may optionally include therapeutic compounds.

Once fluid is communicated to the hub 210 via the fluid port 214, the fluid may flow through the fluid port 214 and into a hollow interior defined by hub 210. The fluid may then flow from the hub 210 and into the sleeve 222 due to the clearance fit defined by the hub 210 and the shaft assembly 220. The fluid may next flow along the longitudinal axis of the sleeve 222 to the open distal end 224, where the fluid may be communicated into an area proximate the window 116 of the endoscope 100. Once the fluid is dispensed into the area proximate the window 116, the fluid may pass over the window 116 thereby cleaning the window 116 of various obstructions.

Also in use, the navigation sensors 226 may be used in combination with the IGS navigation system 50 to navigate the endoscope 100 via the cleaning sleeve 200. Such navigation may be used either during fluid communication by the fluid source 250 or while the fluid source 250 is not in use. Regardless, one or more of the navigation sensors 226 may be used with the IGS navigation system 50 to navigate the endoscope 100 through one or more anatomical passageways of a patient and/or relative to one or more anatomical features of a patient. As described above, such navigation may be combined with navigation of other instruments (e.g., probes) to facilitate real-time overlays of instrument positioning on visualizations produced using the endoscope 100. In some uses, such real-time overlays may be used to facilitate true anatomical measurements of patient anatomy, thereby improving procedure workflows and procedure outcomes.

### III. Examples of Alternative Cleaning Sleeves

As described above, a cleaning sleeve similar to cleaning the sleeve 200 may include a hub configured to engage an endoscope or other instrument such as the endoscope 100. Such hubs may include specific features configured to engage with corresponding components of the endoscope or other instrument. However, such endoscopes or other instruments may include different corresponding engagement features between different instruments. Differing corresponding engagement features may be driven by endoscopes or other instruments produced by different manufactures, endoscopes or other instruments developed for different procedures or use cases, and/or etc. Thus, it may be desirable to include certain features in a given cleaning sleeve to facilitate compatibility with a variety of endoscopes or other instruments despite being produced by different manufacturers or for different procedures or use cases.

A. Example of Alternative Cleaning Sleeve with Interchangeable Cap Feature

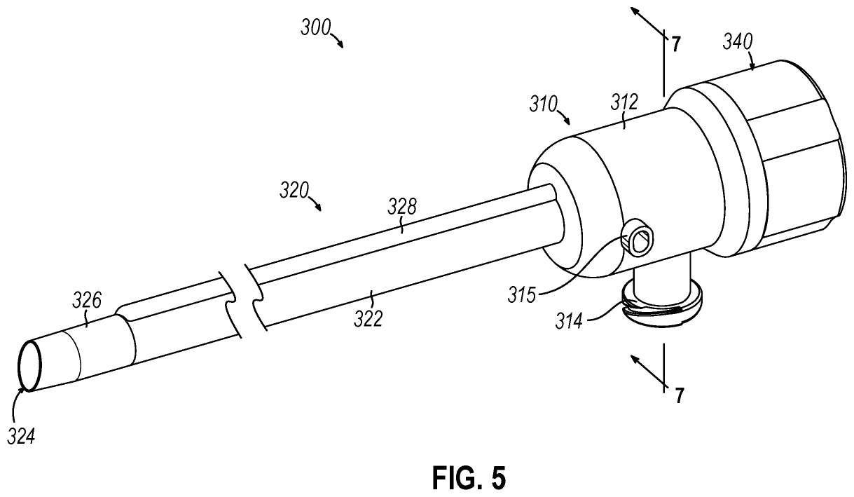

FIG. 5 shows an alternative cleaning sleeve 300 configured for use with the endoscope 100 described above in-lieu of the cleaning sleeve 200. Unless otherwise noted herein, the cleaning sleeve 300 of the present example is substantially similar to the cleaning sleeve 200 described above. For instance, like the cleaning sleeve 200, the cleaning sleeve 300 of the present version includes a shaft assembly 320 extending distally from a hub 310. Additionally, although not shown, it should be understood that the cleaning sleeve 300 may likewise include a cable and connector, which may be substantially similar to the cable 202 and the connector 204 described above. The cable and the connector of the present example are both substantially similar to corresponding structures described above such that additional details are omitted herein.

As with the cleaning sleeve 200 described above, the cleaning sleeve 300 of the present example is configured to direct a flow of fluid through the hub 310 and into the shaft assembly 320 to communicate the fluid to the area proximate the window 116. In other words, the cleaning sleeve 300 is configured to communicate fluid to the area proximate the window 116 to clear obstructions from the window 116 such as tissue, debris, mucous, blood, or other obstructions that may be present during a procedure.

As with the shaft assembly 220 described above, the shaft assembly 320 of the present example includes a sleeve 322 extending distally from the hub 310 and terminating in an open distal end 324. As with the sleeve 222 described above, the sleeve 322 of the present example defines a length less than the length of the shaft 114 of the endoscope 100 such that the window 116 may protrude from the open distal end 324. The sleeve 322 likewise is generally of a rigid configuration but may optionally be configured with at least some flexibility. Although the shaft assembly 320 of the present example is configured with only one sleeve 322, it should be understood that in other examples, the shaft assembly 320 may optionally include multiple sleeves layered together.

As with the shaft assembly 220 described above, the shaft assembly 320 of the present example further includes one or more navigation sensors 326. As described above, incorporation of the navigation sensors 326 may be desirable to add navigation functionality to the endoscope 100 via the cleaning sleeve 300 and the IGS navigation system 50. In the present example, at least one navigation sensor 326 is positioned proximate the open distal end 324. Such positioning may be desirable to facilitate localization of the window 116 due to the proximate relationship between the open distal end 324, the navigation sensor 326, and the window 116. Although not shown, it should be understood that in other examples, additional navigation sensors 326 may be included at various positions along the length of the sleeve 322. As with the navigation sensors 226 described above, the navigation sensors 326 may be coupled to the sleeve 322 in a variety of ways such as sticker-like sensors that may be adhered to the interior or exterior of the sleeve 322, layered between two sleeves and secured via adhesive, friction, or both, and/or integral with a portion of the sleeve 322.

Unlike the shaft assembly 220 described above, the shaft assembly 320 of the present example includes an electrical communication structure 328 integral with the sleeve 322. The electrical communication structure 328 is generally configured to provide a path for electrical communication from the navigation sensors 326 to the hub 310, which may ultimately permit communication with structures similar to the cable 202 and/or the connector 204 described above. In the present version, the electrical communication structure 328 is configured as an elongated bump outwardly from an outer surface of the sleeve 322 and longitudinally relative to the longitudinal axis of the sleeve 322. Although not shown, it should be understood that the interior of the electrical communication structure 328 may include one or more discrete lumens to permit the extension of one or more wires or other electrical communication features along the length of the sleeve 322. In other versions, the electrical communication structure 328 may be configured as an entirely solid component with one or more wires or other electrical communication features embedded within the structure of the electrical communication structure 328.

The hub 310 is disposed on a proximal end of the shaft assembly 320 and is generally configured to facilitate various components of the instrument 100, and other features associated with the cleaning sleeve 300 to interface with the shaft assembly 320. As with the hub 210 described above, the hub 310 of the present example includes a body 312 and a fluid port 314 extending outwardly from the body 312. Although the hub 310 of the present example is shown as including a single fluid port 314, it should be understood that in other examples, multiple fluid ports 314 may be used such as in examples of a hub described in greater detail below. Additionally, the hub 310 includes a wire port 315 extending from a surface of the body 312. The wire port 315 is configured to facilitate electrical communication between structures such as the cable 202 described above and the navigation sensors 326 via communication with at least a portion of the electrical communication structure 328.

Unlike the hub 210 described above, the hub 310 of the present version includes an interchangeable cap 340 removably secured to a proximal end of the body 312. Although the present version is shown with a particular interchangeable cap 340, it should be understood that a variety of interchangeable caps having different configurations may be used with the hub 310. For instance, and as will be described in greater detail below, the interchangeable cap 340 is generally configured to adapt the hub 310 for attachment to specific features of the endoscope 100. Thus, in circumstances where a different endoscope or other instrument is used in combination with the cleaning sleeve 300, a different interchangeable cap 340 may likewise be used to adapt the cleaning sleeve 300 for use with such alternative endoscopes or other instruments.

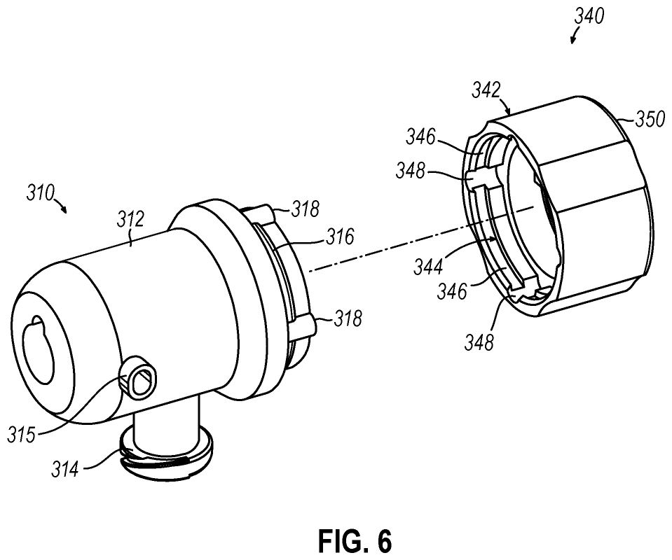

As best seen in FIGS. 6-9, the interchangeable cap 340 includes a distal attachment end 342 and a proximal attachment end 350. As will be understood, the distal attachment end 342 is configured to facilitate removable coupling of the interchangeable cap 340 to the body 312, while the proximal attachment end 350 is configured to facilitate removable coupling of the interchangeable cap 340 to a portion of the endoscope 100. The interchangeable cap 340 is generally of a single material having at least some rigidity and at least some flexibility. The particular balance between rigidity and flexibility (e.g., durometer) may be a function of the particular couplings used with the distal attachment end 342 or the proximal attachment end 350. For instance, some couplings such as snap fits may be better suited for application with more flexible structures, while other couplings such as mechanical locks may be better suited for more rigid structures. Alternatively, in some examples, the interchangeable cap 340 may include one or more overmolded or secondary components to provide localized flexibility with an otherwise rigid structure.

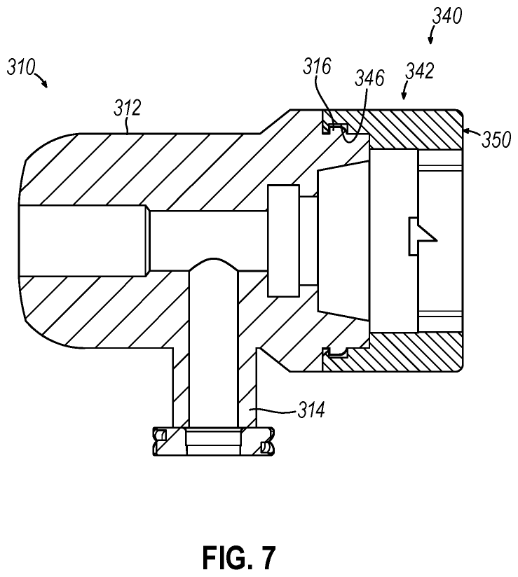

As best seen in FIGS. 6 and 7, the distal attachment end 342 defines an open distal end 344, one or more fasteners 346, and one or more of locator 348. The open distal end 344, the fasteners 346, and the locators 348 are generally configured to receive the proximal end of body 312 and engage corresponding features of the proximal end of body 312 to removably secure the interchangeable cap 340 to the body 312. The fasteners 346 in the present example are configured as a plurality of radially oriented protrusions extending into the interior of the open distal end 344. The fasteners 346 are configured to engage corresponding fasteners 316 of the body 312 to removably couple the interchangeable cap 340 to the body 312 with a snap-fit coupling. Although a snap-fit coupling is used in the present example, it should be understood that other coupling features may be used in other examples. In such examples, the particular configuration of the fasteners 316, 346 may be varied to provide various complementary coupling features between the body 312 and the interchangeable cap 340.

The locators 348 are generally configured to rotationally locate the interchangeable cap 340 relative to the body 312. In particular, the locators 348 of the present version include a plurality of longitudinally oriented recesses extending into the interior of the open distal end 344. Each of the locators 348 is configured to engage a corresponding locator 318 of the body 312 that is complementary to the configuration of the locator 348 of the interchangeable cap 340. Thus, each of the locators 348 is configured to permit attachment of the interchangeable cap 340 to the body 312 in one or more predetermined rotational orientations and to prevent rotation of the interchangeable cap 340 once attached to the body 312. Although particular complementary features are shown for the locators 318, 348, it should be understood that in other examples, alternative complementary features may be used as will be apparent to those of ordinary skill in the art in view of the teachings herein.

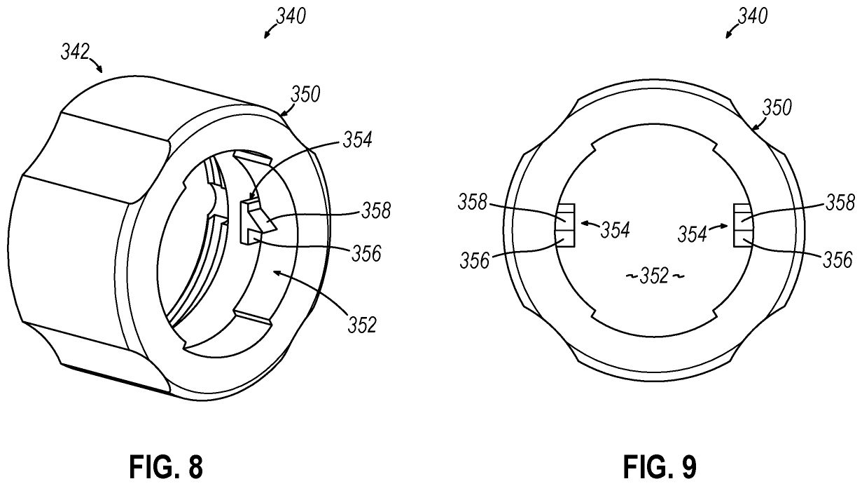

As best seen in FIGS. 8 and 9, the proximal attachment end 350 defines an open proximal end 352 and one or more attachment features 354. The open proximal end 352 is generally configured to receive a portion of the endoscope 100 to permit the attachment features 354 to removably couple to the accessory attachment portion 118 of the endoscope 100. It should be understood that the proximal attachment end 350 is configured to specifically removably couple to the endoscope 100. Thus, the features of the proximal attachment end 350 are a function of the features of the accessory attachment portion 118 of the endoscope 100. In examples where a different endoscope or other instrument is used, the interchangeable cap 340 may be replaced with a different interchangeable cap with a proximal attachment end specific for the different endoscope or other instrument. In other words, the particular proximal attachment end 350 shown and described herein is merely one example and in other examples, a plurality of interchangeable caps 340 of different proximal attachment end configurations may be used to adapt the hub 310 for use with a particular endoscope or other instrument.

The attachment features 354 of the present version are generally configured to releasably engage corresponding features of the accessory attachment portion 118 of the endoscope 100. As described above, the accessory attachment portion 118 may define a flanged configuration, thus the attachment features 354 are configured to releasably engage corresponding flange features. Thus, each of the attachment feature 354 of the present example includes a lock portion 356 extending laterally into the interior of the open proximal end 352. Each of the lock portions 356 may include a flat distal face, which may engage a corresponding flange feature of the accessory attachment portion 118 of the endoscope 100.

Each of the attachment features 354 may optionally include a force applicator 358. In examples including the force applicator 358, the force applicator 358 may be configured to apply a force to a portion of the accessory attachment portion 118 to hold the proximal attachment end 350 in position on the accessory attachment portion 118. In the present example, each of the force applicators 358 is configured as a triangular or ramped feature extending proximally from each of the corresponding attachment features 354. As noted above, the interchangeable cap 340 may have at least some flexibility. Thus, each of the force applicators 358 may compress between the attachment feature 354 and a portion of the accessory attachment portion 118 to apply a force to the accessory attachment portion 118. Such a force may be beneficial to increase friction between the lock portion 356 and the accessory attachment portion 118 to thereby maintain coupling between the proximal attachment end 350 and the accessory attachment portion 118.

In use, the cleaning sleeve 300 may be included with a plurality of different interchangeable caps similar to and/or including the interchangeable cap 340. Each different interchangeable cap may include a varying proximal attachment end with attachment features corresponding to the attachment features of particular endoscopes or other instruments. In the present example, the interchangeable cap 340 may first be attached to the accessory attachment portion 118 of the endoscope 100. Once the interchangeable cap 340 is attached to the endoscope 100, the body 312 may be attached to the interchangeable cap 340. Alternatively, in other uses, the interchangeable cap 340 may be attached to the body 312 first and then the combination of the interchangeable cap 340 and the body 312 may be attached to the endoscope 100.

B. Example of Alternative Cleaning Sleeve with Universal Attachment

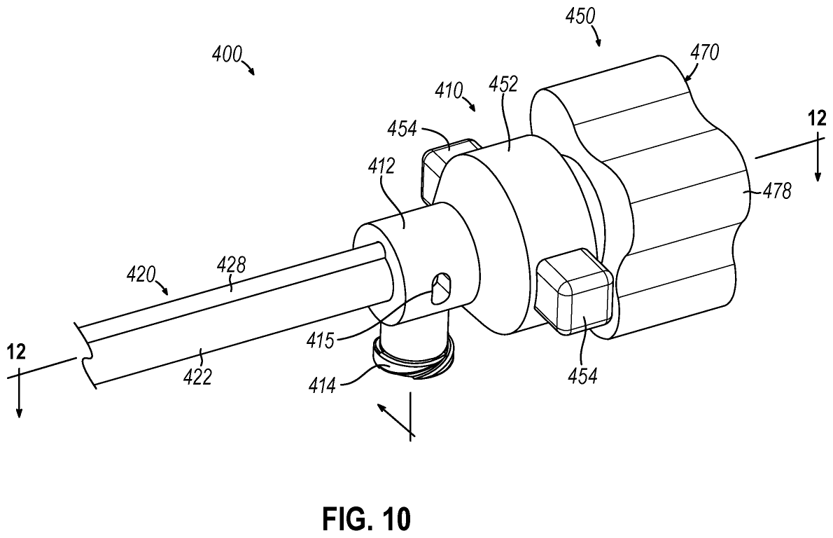

FIG. 10 shows an alternative cleaning sleeve 400 configured for use with the endoscope 100 described above in-lieu of the cleaning sleeve 200. Unless otherwise noted herein, the cleaning sleeve 400 of the present example is substantially similar to the cleaning sleeve 200 described above. For instance, like the cleaning sleeve 200, the cleaning sleeve 400 of the present version includes a shaft assembly 420 extending distally from a hub 410. Additionally, although not shown, it should be understood that the cleaning sleeve 400 may likewise include a cable and connector, which may be substantially similar to the cable 202 and the connector 204 described above. The cable and the connector of the present example are both substantially similar to corresponding structures described above such that additional details are omitted herein.

As with the cleaning sleeve 200 described above, the cleaning sleeve 400 of the present example is configured to direct a flow of fluid through the hub 410 and into the shaft assembly 420 to communicate the fluid to the area proximate the window 116. In other words, the cleaning sleeve 400 is configured to communicate fluid to the area proximate the window 116 to clear obstructions from the window 116 such as tissue, debris, mucous, blood, or other obstructions that may be present during a procedure.

As with the shaft assembly 220 described above, the shaft assembly 420 of the present example includes a sleeve 422 extending distally from the hub 410 and terminating in an open distal end (not shown). As with the sleeve 222 described above, the sleeve 422 of the present example defines a length less than the length of the shaft 114 of the endoscope 100 such that the window 116 may protrude from the open distal end. The sleeve 422 likewise is generally of a rigid configuration but may optionally be configured with at least some flexibility. Although the shaft assembly 420 of the present example is configured with only one sleeve 422, it should be understood that in other examples, the shaft assembly 420 may optionally include multiple sleeves layered together.

As with the shaft assembly 220 described above, the shaft assembly 420 may additionally include one or more navigation sensors (not shown). As described above, incorporation of the navigation sensors may be desirable to add navigation functionality to the endoscope 100 via the cleaning sleeve 400 and the IGS navigation system 50. As similarly described above, at least one navigation sensor may be positioned proximate the open distal end. Such positioning may be desirable to facilitate localization of the window 116 due to the proximate relationship between the open distal end, the navigation sensor, and the window 116. In other examples, additional navigation sensors may be included at various positions along the length of the sleeve 422. As with the navigation sensors 226 described above, the navigation sensors may be coupled to the sleeve 422 in a variety of ways such as sticker-like sensors that may be adhered to the interior or exterior of the sleeve 422, layered between two sleeves and secured via adhesive, friction, or both, and/or integral with a portion of the sleeve 422.

Unlike the shaft assembly 220 described above, the shaft assembly 420 of the present example includes an electrical communication structure 428 integral with the sleeve 422. The electrical communication structure 428 is generally configured to provide a path for electrical communication from one or more navigation sensors to the hub 410, which may ultimately permit communication with structures similar to the cable 202 and/or the connector 204 described above. In the present version, the electrical communication structure 428 is configured as an elongated bump outwardly from an outer surface of the sleeve 422 and longitudinally relative to the longitudinal axis of the sleeve 422. Although not shown, it should be understood that the interior of the electrical communication structure 428 may include one or more discrete lumens to permit the extension of one or more wires or other electrical communication features along the length of the sleeve 422. In other versions, the electrical communication structure 428 may be configured as an entirely solid component with one or more wires or other electrical communication features embedded within the structure of the electrical communication structure 428.

The hub 410 is disposed on a proximal end of the shaft assembly 420 and is generally configured to facilitate various components of the instrument 100, and other features associated with the cleaning sleeve 400 to interface with the shaft assembly 420. As with the hub 210 described above, the hub 410 of the present example includes a body 412 and a fluid port 414 extending outwardly from the body 412. Although the hub 410 of the present example is shown as including a single fluid port 414, it should be understood that in other examples, multiple fluid ports 414 may be used such as in examples of a hub described in greater detail below. Additionally, the hub 410 includes a wire port 415 extending from a surface of body 412. The wire port 415 is configured to facilitate electrical communication between structures such as the cable 202 described above and the navigation sensors via communication with at least a portion of the electrical communication structure 428.

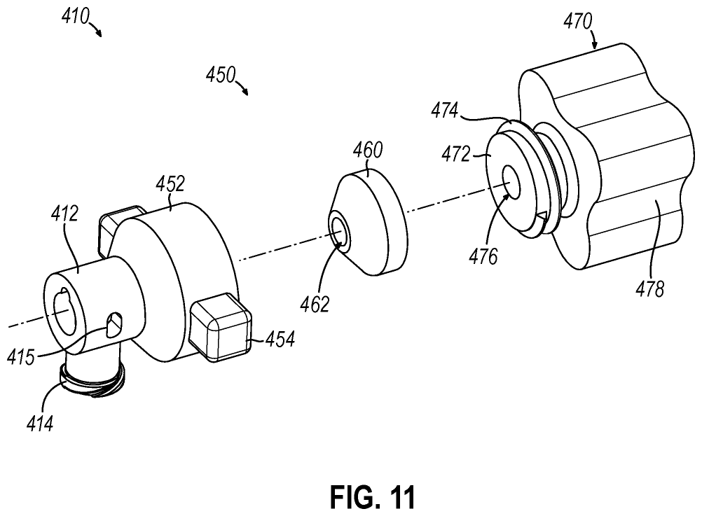

Unlike the hub 210 described above, the hub 410 of the present example additionally includes a universal attachment assembly 450. The universal attachment assembly 450 is generally configured to permit attachment of the hub 410 to a variety of endoscopes or other instruments besides just the endoscope 100. Additionally, the universal attachment assembly 450 may be generally configured to provide flow adjustment for suction used with the cleaning sleeve 400 and the endoscope 100, other endoscopes, or other instruments. As best seen in FIG. 11, the universal attachment assembly 450 includes a receiving collar 452, a flexible seal 460, and a cap 470. Generally, cap 470 is movable relative to the receiving collar 452 to manipulate the configuration of the flexible seal 460 to fasten the hub 410 to the shaft 114 of the endoscope 100 or shafts of other endoscopes or instruments.

The receiving collar 452 extends proximally from the base 412 and is generally of integral construction with the base 412. The receiving collar 452 is generally configured to receive the flexible seal 460 and at least a portion of the cap 470. Thus, the receiving collar 452 may be configured with a larger diameter relative to a diameter of the base 414 to provide additional clearance to receive the flexible seal 460 and/or a portion of the cap 470.

The exterior of the receiving collar 452 includes one or more manipulation protrusions 454. Meanwhile, the interior of the receiving collar 452 includes threading 456 configured to engage corresponding features of the cap 470. Thus, the manipulation protrusions 454 are generally configured to permit manipulation of the receiving collar 452 relative to the cap 470 to fasten the cap 470 to the receiving collar 452 via the threading 456.

The flexible seal 460 is generally positioned within the receiving collar 452 between a portion of the receiving collar 452 and the cap 470. The flexible seal 460 is of a generally tapered disk-shape and defines a through bore 462 extending through the center of the flexible seal 460. The through bore 462 is generally configured to receive the shaft 114 of the endoscope 100. The flexible seal 460 is formed of a generally flexible material such as silicone. As will be described in greater detail below, such flexibility may be desirable to control the size of the through bore 462 to permit fastening of the universal attachment assembly 450 to the shaft 114 of the endoscope 100.

The cap 470 is generally configured to compress the flexible seal 460 between a portion of the cap 470 and the receiving collar 452 to selectively manipulate the size of the through bore 462 of the flexible seal 460. The cap 470 includes a distal receiving end 472 and a proximal manipulation end 478. The receiving end 472 is generally configured for receipt into the receiving collar 452 for removable coupling between the cap 470 and the receiving collar 452.

The receiving end 472 includes threading 474 and a through bore 476 extending longitudinally though the cap 470. The threading 474 is generally complementary to the threading 456 of the receiving collar 452. Thus, the threading 474 may engage the threading 456 of the receiving collar 452 to releasably fasten the cap 470 to the receiving collar 452. Meanwhile, the bore 476 is configured to receive the shaft 114 of the endoscope 100 such that the shaft 114 may pass entirely through the cap 470. Although the through bore 476 of the present example is shown as having a particular diameter, it should be understood that the through bore 476 may be configured with a variety of diameters to accommodate differently sized shafts for use with different endoscopes or other instruments.

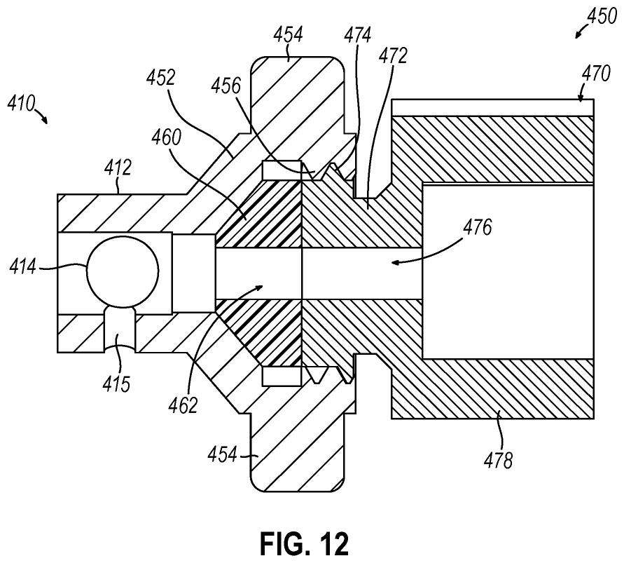

As seen in FIG. 12, in use, the cap 470 may be removably fastened to the receiving collar 452 via the respective threading 456, 474. Such fastening may be achieved by rotating the cap 470 relative to the receiving collar 452. In some uses, the manipulation protrusions 454 and/or the particular shape of the cap 470 may be beneficial to provide additional leverage for rotation of the cap 470. Meanwhile, the flexible seal 460 may be disposed between the receiving collar 452 and the cap 470. Specifically, as the cap 470 is rotated and the threading 456, 474 is engaged, a compressive force may be applied to the flexible seal 460. This compressive force may act on the flexible seal 460 to vary the size of the through bore 462 as a function of the particular compressive force applied. Thus, rotation of the cap 470 may be used to adjust the size of the through bore 462 to removably couple the seal 460 to shaft 114 of the endoscope 100 or another differently sized shaft of another endoscope or other instrument.

In addition, or in the alternative, the combination of the receiving collar 452, the flexible seal 460, and the cap 470 may be used to control the flow of fluid communicated through the hub 410. For instance, in some uses of the cleaning sleeve 400, suction may be applied to the cleaning sleeve 400 similarly to fluid applied to the cleaning sleeve 400 via the fluid port 414. In such versions, such suction may be communicated through the flexible seal 460 at the interface between the flexible seal 460 and the shaft 114 of the endoscope 100. Thus, by controlling the size of the through bore 462, the flow of suction through the flexible seal 460 may also be controlled.

C. Example of Alternative Cleaning Sleeve with Locking Features

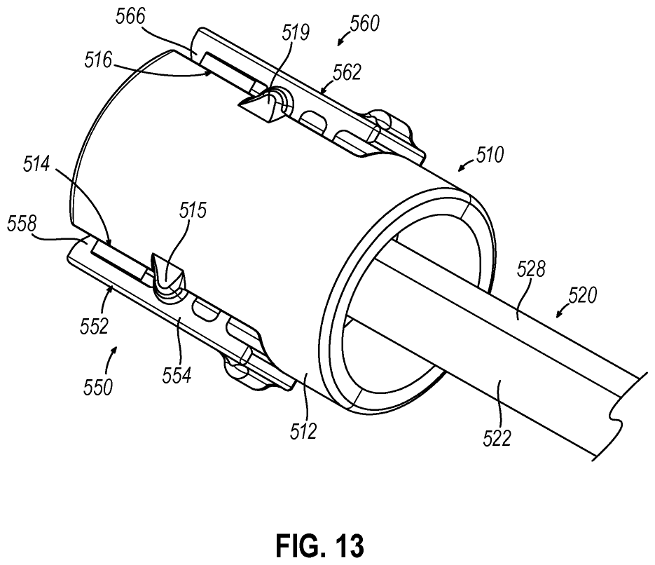

FIG. 13 shows an alternative cleaning sleeve 500 configured for use with the endoscope 100 described above in-lieu of the cleaning sleeve 200. Unless otherwise noted herein, the cleaning sleeve 500 of the present example is substantially similar to the cleaning sleeve 200 described above. For instance, like the cleaning sleeve 200, the cleaning sleeve 500 of the present version includes a shaft assembly 520 extending distally from a hub 510. Additionally, although not shown, it should be understood that the cleaning sleeve 500 may likewise include a cable and a connector, which may be substantially similar to the cable 202 and the connector 204 described above. The cable and the connector of the present example are both substantially similar to corresponding structures described above such that additional details are omitted herein.

As with the cleaning sleeve 200 described above, the cleaning sleeve 500 of the present example is configured to direct a flow of fluid through the hub 510 and into the shaft assembly 520 to communicate the fluid to the area proximate the window 116. In other words, the cleaning sleeve 500 is configured to communicate fluid to the area proximate the window 116 to clear obstructions from the window 116 such as tissue, debris, mucous, blood, or other obstructions that may be present during a procedure.

As with the shaft assembly 220 described above, the shaft assembly 520 of the present example includes a sleeve 522 extending distally from the hub 510 and terminating in an open distal end (not shown). As with the sleeve 222 described above, the sleeve 522 of the present example defines a length less than the length of the shaft 114 of the endoscope 100 such that the window 116 may protrude from the open distal end. The sleeve 522 likewise is generally of a rigid configuration but may optionally be configured with at least some flexibility. Although the shaft assembly 520 of the present example is configured with only one sleeve 522, it should be understood that in other examples, the shaft assembly 520 may optionally include multiple sleeves layered together.

As with the shaft assembly 220 described above, the shaft assembly 520 may additionally include one or more navigation sensors (not shown). As described above, incorporation of one or more navigation sensors may be desirable to add navigation functionality to the endoscope 100 via the cleaning sleeve 500 and the IGS navigation system 50. As similarly described above, at least one navigation sensor may be positioned proximate the open distal end. Such positioning may be desirable to facilitate localization of the window 116 due to the proximate relationship between the open distal end, the navigation sensor, and the window 116. In other examples, additional navigation sensors may be included at various positions along the length of the sleeve 522. As with navigation sensors 226 described above, the navigation sensors may be coupled to the sleeve 522 in a variety of ways such as sticker-like sensors that may be adhered to the interior or exterior of the sleeve 522, layered between two sleeves and secured via adhesive, friction, or both, and/or integral with a portion of the sleeve 522.

Unlike the shaft assembly 220 described above, the shaft assembly 520 of the present example includes an electrical communication structure 528 integral with the sleeve 522. The electrical communication structure 528 is generally configured to provide a path for electrical communication from one or more navigation sensors to the hub 510, which may ultimately permit communication with structures similar to the cable 202 and/or the connector 204 described above. In the present version, the electrical communication structure 528 is configured as an elongated bump outwardly from an outer surface of the sleeve 522 and longitudinally relative to the longitudinal axis of the sleeve 522. Although not shown, it should be understood that the interior of the electrical communication structure 528 may include one or more discrete lumens to permit the extension of one or more wires or other electrical communication features along the length of the sleeve 522. In other versions, the electrical communication structure 528 may be configured as an entirely solid component with one or more wires or other electrical communication features embedded within the structure of the electrical communication structure 528.

The hub 510 is disposed on a proximal end of the shaft assembly 520 and is generally configured to facilitate various components of the instrument 100, and other features associated with the cleaning sleeve 500 to interface with the shaft assembly 520. As with the hub 210 described above, the hub 510 of the present example includes a body 512. Although not shown, it should be understood that the body 512 may optionally include one or more fluid ports and/or wire ports similar to the fluid port 212 and the wire ports 312, 412 described above. As similarly described above, such fluid ports may be configured to communicate fluid from the interior of the hub 510 to the shaft assembly 520. As also similarly described above, such wire ports may be configured to facilitate electrical communication between structures such as the cable 202 described above and the navigation sensors via communication with at least a portion of the electrical communication structure 428.

Unlike the hub 210 described above, the hub 510 of the present example additionally includes an attachment assembly 550 configured to removably secure the hub 510 to the endoscope 100, another different endoscope or other instruments. The attachment assembly 550 of the present example is generally configured as a mechanical latch assembly with one or more features to selectively grip one or more portions of an endoscope or other instruments. In particular, the attachment assembly 550 includes one or more latches 552, 560 movably secured to a portion of the hub 510. In the present example, the attachment assembly 550 includes a first latch 552 and a second latch 560 positioned on opposing sides of the hub 510. In other examples, the attachment assembly 550 may include other suitable number of the latches 552, 560 such as one, three, or more.

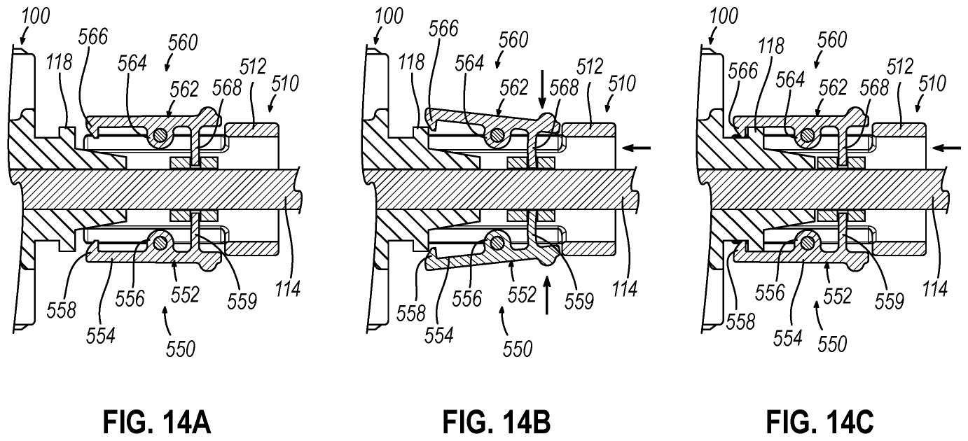

As best seen in FIG. 14A, each latch 552, 560 includes an armature 554, 562 defining a pivot 556, 564 disposed between an engagement feature 558, 566 and a return feature 559, 568. The armature 554, 562 is generally configured as an elongate structure with a length configured to provide sufficient leverage to move the engagement feature 558, 566 via a portion of the return feature 559, 568.

The armature 554, 562 defines the pivot 556, 564 in a portion thereof. The pivot 556, 564 is configured as a partially circular or D-shaped structure and is configured to rotatably receive a portion of the body 512, as will be described in greater detail below. In the present example, the pivot 556, 564 is offset towards one side of the armature 554, 562 to provide increased mechanical advantage or leverage. In particular, the pivot 556, 664 is positioned on the armature 554, 562 closer to the return feature 559, 568 and further from the engagement feature 558, 566. As will be understood, this configuration may be desirable to provide greater mechanical advantage or leverage when driving the engagement feature 558, 566 via the return feature 559, 568 or the distal end of the armature 554, 562.

The engagement feature 558, 566 in the present example is configured as a locking finger or latch tooth, although other suitable structures may be used. Thus, at least a portion the engagement feature 558, 566 extends inwardly towards the center of the body 512 away from the armature 554, 562 to define a finger-shaped or tooth-shaped structure. As will be described in greater detail below, such a finger-shaped or tooth-shaped structure may be configured to receive a portion of the endoscope 100 to selectively couple the hub 510 to the endoscope 100.

The return feature 559, 568 likewise extends inwardly from a the armature 554, 562 toward the center of the body 512. The extension of the return feature 559, 568 is oriented at about 90 degrees relative to the extension of the armature 554, 562, although other suitable orientations may be used. As will be described in greater detail below, the return feature 559, 568 is configured has an elongated structure having at least some resilience or may be resiliently biased to bias the latch 552, 560 from an open configuration to a locked configuration. Thus, the thickness defined by the return feature 559, 568 is generally less than the thickness defined by the armature 554, 562 to provide greater flexibility within the return feature 559, 568 relative to the armature 554, 562, which may be generally rigid. It should be understood that the return feature 559, 568 is entirely optional and may be omitted in some examples.

Each of the latches 552, 560 is rotatable or pivotably secured to a portion of the body 512. In particular, the body 512 defines one or more slots 514, 515 and one or more associated posts 516, 519 corresponding to each of the latches 552, 560. Thus, as the present example includes two latches 552, 560, the body 512 likewise defines two corresponding slots 514, 515 and two corresponding posts 516, 519. In the present configuration, the slots 514, 515 and the posts 516, 519 are positioned opposite each other to orient the laches 552, 560 also opposite each other. The slots 514, 516 are configured to receive a respective latch 552, 560, while the posts 516, 519 are configured to engage a respective pivot 556, 564 to permit movement of each latch into the hollow interior of the body 512 and relative to the body 512. Additionally, each of the slots 514, 516 is generally open to the proximal end of the body 512 to permit receipt of a portion of the endoscope 100 therein, although such an open configuration is entirely optional and may be omitted in some examples.

FIGS. 14A-14C show an example use of the hub 510 in combination with the endoscope 100. Although the present use is shown in the context of use with the endoscope 100, it should be understood that the same use may be readily applied to different endoscopes or other instruments. Indeed, the attachment assembly 550 of the present example is generally configured to provide greater flexibility in coupling the hub 510 to a variety of different coupling features such as those in the endoscope 100, different endoscopes or other instruments entirely.

As best seen in FIG. 14A, the cleaning sleeve 500 may initially be inserted onto the endoscope 100. In particular, the hub 510 may be initially positioned on the shaft 114 of the endoscope 100. In this position, the latches 552, 560 may be positioned proximate the accessory attachment portion 118 with the shaft assembly 520 extending distally away from the latches 552, 560 and the accessory attachment portion 118. Optionally, the cleaning sleeve 500 may additionally be positioned in a desired rotational alignment relative to the endoscope 100, if a particular rotational alignment is desired. Regardless, at this stage, the latches 552, 560 are generally in a relaxed or closed configuration with an orientation generally parallel to the outer surface of the body 512.

Once the cleaning sleeve 500 is inserted onto the endoscope 100, the hub 510 may be coupled to the accessory attachment portion 118 of the endoscope 100 via the attachment assembly 550. As best seen in FIG. 14B, the latches 552, 560 may be transitioned from the relaxed configuration to a flexed or open configuration to prepare the latches 552, 560 for receipt of the accessory attachment portion 118. In this configuration, an inwardly oriented force may be applied to the exterior of each of the latches 552, 560 proximate the return feature 559, 568 and/or the distal end of the armature 554, 562. Such a force may rotate each of the armatures 554, 562 about the pivots 556, 564 to move the engagement features 558, 566 outwardly while the return features 559, 568 move inwardly. During such movement, the return features 559, 568 may engage the shaft 114 of the endoscope 100, thereby deforming the return features 559, 568 and providing some opposing force or resistance to the applied force.

The applied force may be applied until a suitable clearance is provided between the engagement features 558, 566 and one or more portions of the accessory attachment portion 118. At this stage, the cleaning sleeve 500 may be advanced proximally relative to the shaft 114 of the endoscope 100. This proximal advancement may advance the engagement features 558, 556 proximally of one or more portions of the accessory attachment portion 118, to ready the attachment assembly 550 for coupling to the accessory attachment portion 118.

Once proximal advancement is complete, the applied force on the latches 552, 560 may be released. The resistance or opposing force applied by the return features 559, 568 may then move the latches 552, 560 back to the relaxed or closed configuration as shown in FIG. 14C. Once the latches 552, 560 are returned to the relaxed or closed configuration, the engagement features 558, 566 may now engage the accessory attachment portion 118. As described above, the accessory attachment portion 118 may include a flange feature. Thus, the finger-shaped or tooth-shaped structure of the engagement features 558, 566 may be complementary to the flange feature of the accessory attachment portion 118, thereby gripping the flange feature and releasably coupling the cleaning sleeve 500 to the endoscope 100.

A procedure may then be performed using the combination of the endoscope 100 and the cleaning sleeve 500. Once the procedure is complete (or at any stage during the procedure), the cleaning sleeve 500 may optionally be removed from the endoscope 100. Removal of the cleaning sleeve 500 may be accomplished using a reverse of the sequence described above. Alternatively, in some uses, the cleaning sleeve 500 may remain coupled to the endoscope 100 at the conclusion of the procedure such that the cleaning sleeve 500 and the endoscope 100 may be discarded or sterilized for reuse together.

IV. Example of Alternative Cleaning Sleeve with Multiple Fluid Paths

In some examples of a cleaning sleeve, such as the cleaning sleeves 200, 300, 400, 500 described above, it may be desirable to use multiple fluid mediums or fluid pressures in connection with such cleaning sleeve. For instance, as described above such a cleaning sleeve may be used to apply fluid to the window of an endoscope similar to the endoscope 100, thereby cleaning the window. Thus, a first fluid may be desirable for cleaning purposes. Additionally, it may also be desirable to remove fluid or other matter via suction. Thus, it may be desirable in some cleaning sleeves to include another fluid path to facilitate the flow of suction or vacuum. On other words, rather than a particular second fluid being used, a second fluid may consist of fluid being withdrawn from the cleaning sleeve via suction or vacuum.

In addition to the above, it may be desirable to introduce a second fluid through such a cleaning sleeve. In other words, it may be desirable to apply the second fluid similarly to the first fluid via a second discrete fluid path in addition to the first fluid. The use of an additional fluid may be desirable to facilitate other functions beyond cleaning. For instance, it may be desirable to use both non-active fluids and active fluids (e.g., therapeutic fluids) during a procedure either simultaneously or independently. Accordingly, in such cleaning sleeves it may be desirable to include a plurality of discrete fluid paths to support the use of multiple fluids either in the form of introduced fluids or fluids withdrawn via suction.

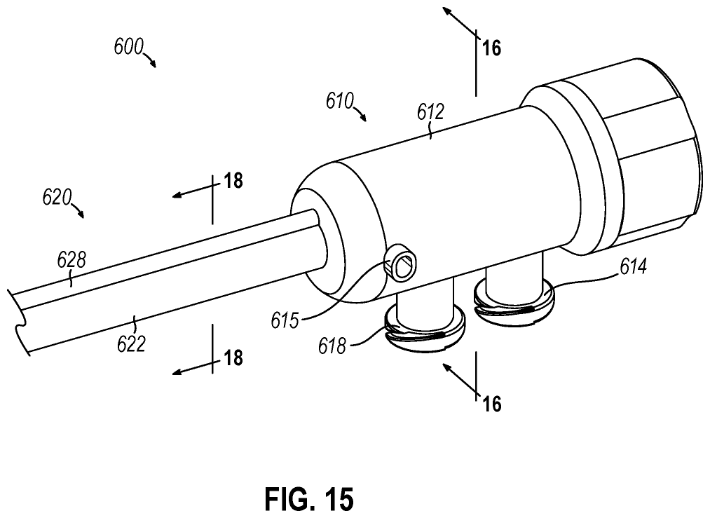

FIG. 15 shows an alternative cleaning sleeve 600 configured for use with the endoscope 100 described above in-lieu of the cleaning sleeve 200. Unless otherwise noted herein, the cleaning sleeve 600 of the present example is substantially similar to the cleaning sleeve 200 described above. For instance, like the cleaning sleeve 200, the cleaning sleeve 600 of the present version includes a shaft assembly 620 extending distally from a hub 610. Additionally, although not shown, it should be understood that the cleaning sleeve 600 may likewise include a cable and a connector, which may be substantially similar to the cable 202 and the connector 204 described above. The cable and the connector of the present example are both substantially similar to corresponding structures described above such that additional details are omitted herein.

As with the cleaning sleeve 200 described above, the cleaning sleeve 600 of the present example is configured to direct a flow of fluid through the hub 610 and into the shaft assembly 620 to communicate the fluid to the area proximate the window 116. In other words, the cleaning sleeve 600 is configured to communicate fluid to the area proximate the window 116 to clear obstructions from the window 116 such as tissue, debris, mucous, blood, or other obstructions that may be present during a procedure.

As with the shaft assembly 220 described above, the shaft assembly 620 of the present example includes a sleeve 622 extending distally from the hub 610 and terminating in an open distal end (not shown). As with the sleeve 222 described above, the sleeve 622 of the present example defines a length less than the length of the shaft 114 of the endoscope 100 such that the window 116 may protrude from the open distal end. The sleeve 622 likewise is generally of a rigid configuration but may optionally be configured with at least some flexibility. Although the shaft assembly 620 of the present example is configured with only one sleeve 622, it should be understood that in other examples, the shaft assembly 620 may optionally include multiple sleeves layered together.

As with the shaft assembly 220 described above, the shaft assembly 620 may additionally include one or more navigation sensors (not shown). As described above, incorporation of one or more navigation sensors may be desirable to add navigation functionality to the endoscope 100 via the cleaning sleeve 600 and the IGS navigation system 50. As similarly described above, at least one navigation sensor may be positioned proximate the open distal end. Such positioning may be desirable to facilitate localization of the window 116 due to the proximate relationship between the open distal end, the navigation sensor, and the window 116. In other examples, additional navigation sensors may be included at various positions along the length of the sleeve 622. As with the navigation sensors 226 described above, the navigation sensors may be coupled to the sleeve 622 in a variety of ways such as sticker-like sensors that may be adhered to the interior or exterior of the sleeve 622, layered between two sleeves and secured via adhesive, friction, or both, and/or integral with a portion of the sleeve 422.

Unlike the shaft assembly 220 described above, s the haft assembly 620 of the present example includes an electrical communication structure 628 integral with the sleeve 622. The electrical communication structure 628 is generally configured to provide a path for electrical communication from one or more navigation sensors to the hub 610, which may ultimately permit communication with structures similar to the cable 202 and/or the connector 204 described above. In the present version, the electrical communication structure 628 is configured as an elongated bump outwardly from an outer surface of the sleeve 622 and longitudinally relative to the longitudinal axis of the sleeve 622. Although not shown, it should be understood that the interior of the electrical communication structure 628 may include one or more discrete lumens to permit the extension of one or more wires or other electrical communication features along the length of the sleeve 622. In other versions, the electrical communication structure 628 may be configured as an entirely solid component with one or more wires or other electrical communication features embedded within the structure of the electrical communication structure 628.

The hub 610 is disposed on a proximal end of the shaft assembly 620 and is generally configured to facilitate various components of the instrument 100, and other features associated with the cleaning sleeve 600 to interface with the shaft assembly 620. As with the hub 210 described above, the hub 610 of the present example includes a body 612 and a fluid port 614 extending outwardly from the body 612. Additionally, the hub 610 includes a wire port 615 extending from a surface of body 612. The wire port 615 is configured to facilitate electrical communication between structures such as the cable 202 described above and the navigation sensors via communication with at least a portion of the electrical communication structure 628. Optionally, the hub 610 may include a cap or other coupling feature similar to the interchangeable cap 340 described above, which may releasably fasten to the body 612.

Unlike the hub 210 described above, the hub 610 of the present example includes multiple fluid ports 614, 618. As will be described in greater detail below, the fluid ports 614, 618 are configured to independently communicate fluid either to or from the sleeve 622 of the shaft assembly 620. The fluid ports 614, 618 include a proximal fluid port 614 and a distal fluid port 618. As will be described in greater detail below, the proximal fluid port 614 is configured to communicate fluid to or from one portion of the sleeve 622 of the shaft assembly 620, while the distal fluid port 618 is configured to communicate fluid to or from another portion of the sleeve 622. Although the fluid ports 614, 618 are shown as being substantially similar in structure, it should be understood that in some examples the particular structure of each of the fluid ports 614, 618 may vary relative to the other. In some examples, at least some variation of the structure of the fluid ports 614, 618 may be desirable promote identification of a particular the fluid ports 614, 618 without direct visualization. In addition, or in the alternative, the fluid ports 614, 618 may be configured to accommodate different fluids and/or fluids with different flow properties. As a result, the physical structure of the fluid ports 614, 618 may vary to accommodate such variation in fluids and/or flow properties.

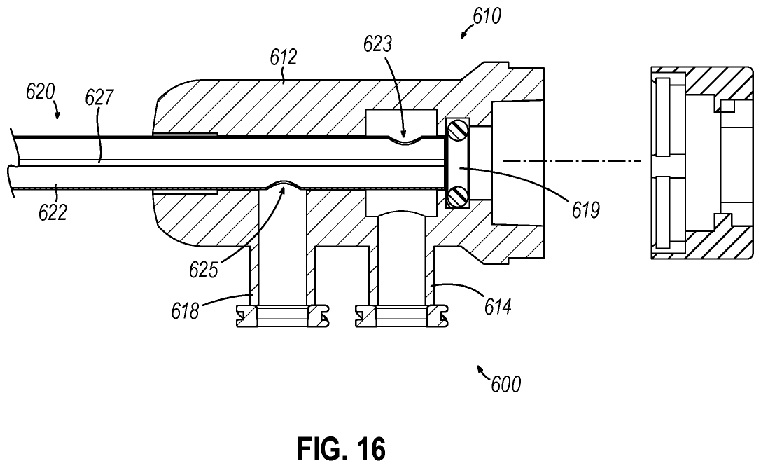

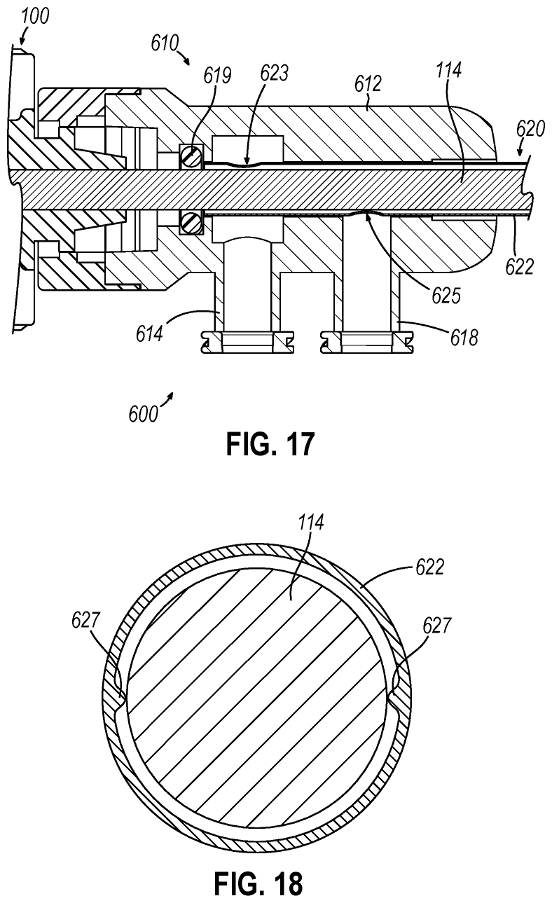

As best seen in FIG. 16, the sleeve 622 may include a distal opening 625 and a proximal opening 623 corresponding to the distal fluid port 618 and the proximal fluid port 614, respectively. As can be seen, the distal opening 625 and the proximal opening 623 are oriented on the sleeve 622 opposite each other. As will be described in greater detail below, this configuration may be desirable to permit the distal opening 625 and the proximal opening 623 to communicate with separate fluid paths defined within the sleeve 622. Although the distal opening 625 is shown as being on the bottom of the sleeve 622 and the proximal opening 623 is shown as being on the top of the sleeve 622, the orientation may be different in other examples. For instance, in some examples, the orientation may be reversed with the distal opening 625 being on the top of the sleeve 622 and the proximal opening 623 being on the bottom of the sleeve 622. In still other examples, alternative orientations may be used such as the distal opening 625 and the proximal opening 623 being oriented on the sides of the sleeve 622. Additionally, the sleeve 622 may optionally abut an o-ring 619 to restrict fluid leakage to other portions of the hub 610.

As described above, the sleeve 622 may define a plurality of discrete fluid flow paths therein for communication of fluids from the fluid ports 614, 618, through the openings 623, 625, into the sleeve 622, and through the sleeve 622. As best seen in FIG. 18, the sleeve 622 includes a plurality of dividers 627 extending into the hollow interior of the sleeve 622. The dividers 627 are generally configured to segregate the interior of the sleeve 622 into discrete fluid flow paths when the sleeve 622 is disposed on a cylindrical structure such as the shaft 114 of endoscope 100.

Each of the dividers 627 is generally configured as a semi-cylindrical protrusion oriented toward the center of the sleeve 622. The length of such an extension or the thickness of each divider is generally configured to permit engagement with the shaft 114 of the endoscope 100 to fluidly isolate one area of the hollow interior of the sleeve 622 from another. Thus, the particular length of extension or the thickness of each of the dividers 627 may generally be a function of the clearance between the sleeve 622 and the shaft 114 of the endoscope 100. To further facilitate fluid isolation, it should be understood that in some examples, each of the dividers 627 may be formed of an elastomeric material either integral with the sleeve 622 or overmolded to a surface of the sleeve 622. Although not shown, it should be understood that each of the dividers 627 may extend longitudinally the entire length of the sleeve 622. Additionally, although the present version is shown as including two dividers 627, it should be understood that additional dividers 627 may be used in other examples to define any suitable number of fluid paths within the sleeve 622.

In use, the cleaning sleeve 600 may be used with the endoscope 100, different endoscopes, or other instruments entirely. During such uses, fluid may be supplied via the proximal fluid port 614 or the distal fluid port 618 simultaneously, independently, or in various combinations thereof. Such fluid may be communicated through a given fluid port 614, 618, through a given opening 623, 625 in the sleeve 622 and through the sleeve 622 via a given discrete fluid paths provided by the dividers 627. Various suitable fluids may be used such as saline or therapeutic fluids. Additionally, or in the alternative, the fluid ports 614, 618 may be used to evacuate fluids using suction or vacuum. In merely one example, one of the fluid ports 614, 618 may be used to supply a fluid such as saline for cleaning purposes, while another of the fluid ports 618, 614 may be used to evacuate fluid from the region surrounding the window 116 using suction or vacuum. Of course, various alternative combinations of fluids and/or suction may be used as will be appreciated by those of ordinary skill in the art in view of the teachings herein.

In another use, the cleaning sleeve 600 may be used without endoscope 100 or any other instruments as a standalone device. In such uses, the cleaning sleeve 600 may be used as a suction device rather than for cleaning. Thus, one or both of the fluid ports 614, 618 may be used to communicate suction. Optionally, one of the fluid ports 614, 618 may be used to supply another fluid for purposes other than cleaning associated with a given procedure.

### V. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus for use with an endoscope, comprising: (a) a hub, the hub being configured to engage a portion of the endoscope, the hub including a fluid port; (b) a shaft assembly extending from the hub and defining a hollow interior, the hollow interior being configured to receive a tubular portion of the endoscope and communicate fluid within the shaft assembly to an open distal end of the shaft assembly; and (c) one or more navigation sensors, at least one of the one or more navigation sensors being disposed proximate the distal end of the shaft assembly, the one or more navigation sensors being configured generate signals indicating a position of at least a portion of the shaft assembly in three dimensional space.

### Example 2

The apparatus of Example 1, the one or more navigation sensors including a plurality of navigation sensors.

### Example 3

The apparatus of Example 2, the one or more navigation sensors include a first navigation sensor and a second navigation sensor, the first navigation sensor being disposed on the shaft assembly proximate the distal end, the second navigation sensor being disposed on the shaft assembly in a different position relative to the first navigation sensor.

### Example 4

The apparatus of any of Examples 2 through 3, further comprising a flexible circuit secured to a portion of the shaft assembly, each navigation sensor of the one or more navigation sensors being in electrical communication with the hub via the flexible circuit.

### Example 5

The apparatus of any of Examples 1 through 3, a portion of the shaft assembly defining an electrical communication structure extending longitudinally along a length of the shaft assembly, the electrical communication structure including one or more wires, each navigation sensor of the one or more navigation sensors being in electrical communication with the hub via the one or more wires.

### Example 6

The apparatus of any of Examples 1 through 5, the hub including a plurality of interchangeable caps, each interchangeable cap being configured to removably couple to the proximal end of the hub to adapt the hub for engagement with a portion of the endoscope.

### Example 7

The apparatus of Example 6, at least one interchangeable cap of the plurality of interchangeable caps including one or more attachment features configured to engage a flanged feature of the endoscope.

### Example 8

The apparatus of any of Examples 1 through 5, the hub including a body and a universal attachment assembly removably secured to the body, a portion of the universal attachment assembly being movable relative to the body to selectively manipulate engagement between the hub and a portion of the endoscope.

### Example 9

The apparatus of Example 7, the universal attachment assembly include a receiving collar and a cap, the cap being disposed between the body and the receiving collar, the cap being flexible and having a through bore, the receiving collar being configured to move relative to the body to compress the cap between the body and the receiving collar, thereby manipulating the shape of the through bore of the cap.

### Example 10

The apparatus of Example 9, the receiving collar including threading, the threading of the receiving collar being configured to engage threading of the body.

### Example 11

The apparatus of any of Examples 1 through 5, the hub further including a body and an attachment assembly, the attachment assembly including one or more latches, each latch being configured to move relative to the body to selectively engage a portion of the endoscope.

### Example 12

The apparatus of Example 11, each latch including an engagement feature and a return feature, the return feature being configured to bias the engagement feature toward a predetermined position.

### Example 13

The apparatus of Examples 11 or 12, the body including one or more posts corresponding to each latch of the one or more latches, each latch including a pivot configured to receive a corresponding post of the one or more posts, each pivot being configured to rotate about the corresponding post.

### Example 14

The apparatus of any of Examples 1 through 13, the hub further including a second fluid port, the first fluid port and the second fluid port both being in communication with the shaft assembly.

### Example 15

The apparatus of Example 14, the shaft assembly defining one or more dividers extending into the hollow interior defined by the shaft assembly, the one or more dividers defining a first fluid path and a second fluid path, the first fluid port being in communication with the first fluid path, the second fluid port being in communication with the second fluid path, at least one of the first fluid port or the second fluid port being configured to communicate suction to the open distal end of the shaft assembly via a respective first fluid path or second fluid path.

### Example 16

An assembly for use in an ENT procedure, the assembly comprising: (a) an endoscope, the endoscope including a body and a shaft extending from the body, the body defining an accessory attachment portion proximate a portion of the shaft, the shaft including a window proximate a distal end of the shaft; and (b) a cleaning sleeve, the cleaning sleeve including: (i) a hub configured to receive a portion of the shaft of the endoscope, the hub including a body and a fluid port extending from the body, (ii) a shaft assembly extending distally from the hub, the shaft assembly including a sleeve configured to receive the shaft of the endoscope within a hollow interior defined by the sleeve, the shaft assembly being configured to define a fluid path between an inner surface of the sleeve and an outer surface of the shaft of the endoscope, the fluid path being in communication with the fluid port of the hub to communicate fluid onto the window of the endoscope from an open distal end of the sleeve, and (iii) one or more navigation sensors secured to a portion of the cleaning sleeve, each navigation sensor being configured to generate signals indicating apposition of one or more features of the cleaning sleeve in three dimensional space.

### Example 17

The assembly of Example 16, the hub including one or more attachment features configured to removably secure the hub to the accessory attachment portion of the endoscope.

### Example 18

The assembly of Example 17, the one or more attachment features including at least one or more of an interchangeable cap, a threaded receiving collar, and a latch arm.

### Example 19

The assembly of any of Examples 16 through 18, the hub further including a suction port extending from the hub, the fluid port and the suction port both being in communication with the fluid path defined between the sleeve and the shaft of the endoscope, the fluid port being configured to communicate liquid to the fluid path, the suction port being configured to communicate suction to the fluid path.

### Example 20

A method of using an endoscope with a cleaning sleeve, the method comprising: (a) inserting a shaft of the endoscope into the cleaning sleeve to align an open distal end of the cleaning sleeve with a window of the shaft of the endoscope; (b) coupling a hub of the cleaning sleeve to at least a portion of the endoscope to removably secure the cleaning sleeve to the endoscope; (c) locating the open distal end of the cleaning sleeve in three dimensional space to navigate the window of the endoscope using one or more navigation sensors coupled to the cleaning sleeve; and (d) communicating a fluid between the cleaning sleeve and the shaft of the endoscope and out of the open distal end to clean at least a portion of the window of the endoscope.

### Example 21

The method of Example 20, further comprising locating at least one other feature of the cleaning sleeve in three dimensional space to navigate one or more elements of the endoscope using the one or more navigation sensors.

### Example 22

The method of any of Examples 20 through 21, the act of coupling the hub of the cleaning sleeve to the at least a portion of the endoscope including: (i) selecting a selected one interchangeable cap from a plurality of interchangeable caps, the selected one interchangeable cap including at least one feature corresponding to an accessory attachment portion of the endoscope, (ii) coupling the selected one interchangeable cap to a portion of the hub, and (iii) coupling the selected one interchangeable cap to the accessory attachment portion of the endoscope.

### Example 23

The method of any of Examples 20 through 21, the act of coupling the hub of the cleaning sleeve to the at least a portion of the endoscope including rotating a receiving collar of the cleaning sleeve relative to a body of the hub to manipulate the configuration of a flexible cap disposed between the receiving collar and the body to thereby secure the flexible cap to the shaft of the endoscope.

### Example 24

The method of any of Examples 20 through 21, the act of coupling the hub of the cleaning sleeve to the at least a portion of the endoscope including rotating a pair of latches of the hub of the cleaning sleeve relative to a body of the cleaning sleeve to coupled a portion of each latch of the pair of latches to an accessory attachment portion of the endoscope.

### Example 25

The method of any of Examples 20 through 24, further comprising communicating suction between the cleaning sleeve and the shaft of the endoscope and through the open distal end to remove fluid from an area proximate the window of the endoscope.

### V. Miscellaneous

It should be understood that any of the teachings, expressions, embodiments, examples, etc. described herein may be combined with any of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one skilled in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. An apparatus for use with an endoscope, comprising:
a hub, the hub (i) being configured to engage a portion of the endoscope, and (ii) including a fluid port,
a shaft assembly extending from the hub and defining a hollow interior, the hollow interior being configured to receive a tubular portion of the endoscope and communicate fluid within the shaft assembly to an open distal end of the shaft assembly, and
one or more navigation sensors, at least one of the one or more navigation sensors being disposed proximate the distal end of the shaft assembly, wherein the one or more navigation sensors being configured generate signals indicating a position of at least a portion of the shaft assembly in three dimensional space.

2. The apparatus of claim 1, wherein the one or more navigation sensors includes a plurality of navigation sensors.

3. The apparatus of claim 2, wherein:
the one or more navigation sensors include a first navigation sensor and a second navigation sensor,
the first navigation sensor being disposed on the shaft assembly proximate the distal end, and
the second navigation sensor being disposed on the shaft assembly in a different position relative to the first navigation sensor.

4. The apparatus of claim 1, further comprising a flexible circuit secured to a portion of the shaft assembly, wherein each navigation sensor of the one or more navigation sensors being in electrical communication with the hub via the flexible circuit.

5. The apparatus of claim 1, wherein:
a portion of the shaft assembly defines an electrical communication structure extending longitudinally along a length of the shaft assembly,
the electrical communication structure includes one or more wires, and
each navigation sensor of the one or more navigation sensors being in electrical communication with the hub via the one or more wires.

6. The apparatus of claim 1, wherein:
the hub including a plurality of interchangeable caps, and
each interchangeable cap being configured to removably couple to the proximal end of the hub to adapt the hub for engagement with a portion of the endoscope.

7. The apparatus of claim 6, wherein at least one interchangeable cap of the plurality of interchangeable caps includes one or more attachment features configured to engage a flanged feature of the endoscope.

8. The apparatus of claim 1, wherein:
the hub includes a body and a universal attachment assembly removably secured to the body, and
a portion of the universal attachment assembly being movable relative to the body to selectively manipulate engagement between the hub and a portion of the endoscope.

9. The apparatus of claim 7, wherein:
the universal attachment assembly includes a receiving collar and a cap,
the cap being disposed between the body and the receiving collar,
the cap being flexible and having a through bore, and
the receiving collar being configured to move relative to the body to compress the cap between the body and the receiving collar thereby manipulating the shape of the through bore of the cap.

10. The apparatus of claim 9, wherein:
the receiving collar includes threading, and
the threading of the receiving collar being configured to engage threading of the body.

11. The apparatus of claim 1, wherein:
the hub further includes a body and an attachment assembly,
the attachment assembly includes one or more latches, and
each latch of the one or more latches being configured to move relative to the body to selectively engage a portion of the endoscope.

12. The apparatus of claim 11, wherein:
each latch of the one or more latches includes an engagement feature and a return feature, and
the return feature being configured to bias the engagement feature toward a predetermined position.

13. The apparatus of claim 11, wherein:
the body includes one or more posts corresponding to each latch of the one or more latches,
each latch of the one or more latches includes a pivot configured to receive a corresponding post of the one or more posts, and
each pivot being configured to rotate about the corresponding post.

14. The apparatus of any of claim 1, wherein:
the hub further includes a second fluid port, and
the first fluid port and the second fluid port both being in communication with the shaft assembly.

15. The apparatus of claim 14, wherein:
the shaft assembly defines one or more dividers extending into the hollow interior defined by the shaft assembly,
the one or more dividers defining a first fluid path and a second fluid path,
the first fluid port being in communication with the first fluid path,
the second fluid port being in communication with the second fluid path, and
at least one of the first fluid port or the second fluid port being configured to communicate suction to the open distal end of the shaft assembly via a respective first fluid path or second fluid path.

16. An assembly for use in an ENT procedure, the assembly comprising:
an endoscope, the endoscope including a body and a shaft extending from the body, the body defining an accessory attachment portion proximate a portion of the shaft, the shaft including a window proximate a distal end of the shaft, and
a cleaning sleeve, the cleaning sleeve including: (i) a hub configured to receive a portion of the shaft of the endoscope, the hub including a body and a fluid port extending from the body, (ii) a shaft assembly extending distally from the hub, the shaft assembly including a sleeve configured to receive the shaft of the endoscope within a hollow interior defined by the sleeve, the shaft assembly being configured to define a fluid path between an inner surface of the sleeve and an outer surface of the shaft of the endoscope, the fluid path being in communication with the fluid port of the hub to communicate fluid onto the window of the endoscope from an open distal end of the sleeve, and (iii) one or more navigation sensors secured to a portion of the cleaning sleeve, each navigation sensor being configured to generate signals indicating apposition of one or more features of the cleaning sleeve in three dimensional space.

17. The assembly of claim 16, wherein the hub includes one or more attachment features configured to removably secure the hub to the accessory attachment portion of the endoscope.

18. The assembly of claim 17, wherein the one or more attachment features includes at least one or more of an interchangeable cap, a threaded receiving collar, and a latch arm.

19. The assembly of claim 16, wherein:
the hub further includes a suction port extending from the hub,
the fluid port and the suction port both being in communication with the fluid path defined between the sleeve and the shaft of the endoscope,
the fluid port being configured to communicate liquid to the fluid path, and
the suction port being configured to communicate suction to the fluid path.

20. A method of using an endoscope with a cleaning sleeve, the method comprising:
inserting a shaft of the endoscope into the cleaning sleeve to align an open distal end of the cleaning sleeve with a window of the shaft of the endoscope,
coupling a hub of the cleaning sleeve to at least a portion of the endoscope to removably secure the cleaning sleeve to the endoscope,
locating the open distal end of the cleaning sleeve in three dimensional space to navigate the window of the endoscope using one or more navigation sensors coupled to the cleaning sleeve, and
communicating a fluid between the cleaning sleeve and the shaft of the endoscope and out of the open distal end to clean at least a portion of the window of the endoscope.

21. The method of claim 20, further comprising locating at least one other feature of the cleaning sleeve in three dimensional space to navigate one or more elements of the endoscope using the one or more navigation sensors.

22. The method of claim 20, wherein coupling the hub of the cleaning sleeve to the at least a portion of the endoscope includes:
selecting a selected one interchangeable cap from a plurality of interchangeable caps, the selected one interchangeable cap including at least one feature corresponding to an accessory attachment portion of the endoscope,
coupling the selected one interchangeable cap to a portion of the hub, and
coupling the selected one interchangeable cap to the accessory attachment portion of the endoscope.

23. The method of claim 20, wherein coupling the hub of the cleaning sleeve to the at least a portion of the endoscope includes rotating a receiving collar of the cleaning sleeve relative to a body of the hub to manipulate the configuration of a flexible cap disposed between the receiving collar and the body to thereby secure the flexible cap to the shaft of the endoscope.

24. The method of claim 20, wherein coupling the hub of the cleaning sleeve to the at least a portion of the endoscope includes rotating a pair of latches of the hub of the cleaning sleeve relative to a body of the cleaning sleeve to coupled a portion of each latch of the pair of latches to an accessory attachment portion of the endoscope.

25. The method of claim 20, further comprising communicating suction between the cleaning sleeve and the shaft of the endoscope and through the open distal end to remove fluid from an area proximate the window of the endoscope.
